(19) United States
(12) Patent Application Publication
(10) Pub. No.: US 2006/0121599 A1
(43) Pub. Date: Jun. 8, 2006

(54) Method for producing a sample carrier for maldi-mass spectrometry

(76) Inventor: Karsten Reihs, Koln (DE)

Correspondence Address: PERMAN & GREEN, 425 POST ROAD, FAIRFIELD, CT, 06824, US

(21) Appl. No.: 10/525,453

(22) PCT Filed: Dec. 15, 2003
(86) PCT No.: PCT/EP03/14230

§ 371 Date: Feb. 18, 2005

(30) Foreign Application Priority Data
Dec. 13, 2002 (DE) 102 58 674.8

(51) Int. Cl. G01N 1/28; H01J 49/00; C12M 1/34; B05D 3/00

(52) U.S. Cl. 435/287.1; 427/2.11; 250/288

(57) ABSTRACT

The invention relates to a method for producing a sample carrier compromising a plurality of MALDI matrix points, a surface material which can be obtained according to the inventive method and a surface material which is stable for a long time.

Process for manufacturing a sample carrier for MALDI mass spectrometry The present invention concerns a process for making a sample carrier with a multitude of MALDI matrix points, a surface formation which, according to the invention, can be obtained in the course of the process, and a surface formation which is stable for a long time.

Mass spectrometry has been implemented more and more for the analysis of samples, for example in the additives industry or in biological research and production. For the purposes of analysing biomolecules in the samples, it is preferred to implement mass spectrometry with ionisation using matrix-assisted laser desorption and ionisation (MALDI).

For the purposes of the MALDI process, especially biomolecules and/or biological material in the form of a drop of liquid are metered, for example with a pipette, on to a so-called MALDI matrix point before they are dried. The crystals formed thereby are examined for example with a MALDI-TOF mass spectrometer in linear or reflector mode. Details of this process can be found in Nordhoff et. al., “MALDI-MS as a new method for the analysis of nucleic acid (DNA and RNA) with molecular masses up to 150,000 Dalton, Application of modern mass spectrometric methods to plant science research,” Oxford University Press, (1996), pages 86-101 which is hereby incorporated as a reference and thus counts as part of the disclosure.

The MALDI matrix points are produced in accordance with the state of the art by applying the matrix substance as a solution in the form of a drop of liquid onto a sample carrier and leaving it there to dry. This process is very time-consuming in spite of automation technology, especially in connection with series tests in which, at times, more than 200 MALDI matrix points have to be applied to the sample carrier. Furthermore, the MALDI matrix points are not regular in shape and are not homogeneous among themselves. In addition, the position of the matrix points on the sample carrier is relatively inexact. In order to prevent two neighbouring points from merging, their spacing must be chosen to be correspondingly large.

Therefore, the task is to provide a process for making a surface formation with a multitude of MALDI matrix points, which does not show the disadvantages of the state of the art.

According to the invention, the task is solved by means of a process for making a surface formation, preferably a sample carrier, with a multitude of MALDI matrix points, during which the MALDI matrix points are applied by precipitation of the MALDI matrix substance from the gas phase onto the sample carrier.

Those of ordinary skill in the art were astonished when they realized that the process according to the invention succeeded in creating any number of MALDI matrix points simultaneously. The MALDI matrix points can have any form. They can be created very reproducibly and homogeneously and can show a surface structure which allows the achievement of very good mass spectrums.

For the purpose of the invention, a surface formation is any shaped body with a surface of any shape. However, the surface formation should ideally be a plate with an even surface, most suitably a sample carrier, which should, however, not have any recesses. Ideally, according to the invention, the surface formation is a film.

For the purpose of the invention, a MALDI matrix point consists, for the most part, of at least one MALDI matrix substance well known to those of ordinary skill in the art. Preferred MALDI matrix substances are 3-hydroxypicolinic acid, α-cyano-4-hydroxycoumarin acid, 2.5 dihydroxybenzoic acid, sinapic acid, 2,4,6-trihydroxyacetophenone nitrobenzyl alcohol, nicotinic acid, ferulic acid, caffeic acid, 2-aminobenzoic acid, picolinic acid, 3-aminobenzoic acid, 2,3,4-trihydroxyacetophenone, 6-aza-2-thiothymine, urea, succinic acid, adipic acid, malonic acid or mixtures thereof. Particularly preferred is the MALDI matrix substance α-cyano-4-hydroxycoumarin acid.

Preferably, a compound which sublimes on the surface formation and is visible to the human eye is selected as the MALDI matrix substance.

According to the invention, the MALDI matrix points are created by precipitation of the MALDI matrix substance from the gas phase. Precipitation from the gas phase for the purpose of the invention is every process during which the MALDI matrix substance is applied from the gas phase to the surface formation. For example, condensation or sublimation are proposed. Preferably, however, the application of the MALDI matrix points to the surface formation is by sublimation. For the purpose of this invention, sublimation comprises the MALDI matrix substance being transferred as a solid material to the gas phase and/or precipitated as solid material on the surface formation from the gas phase. Preferably, the sublimation takes place in a vacuum. Especially preferred is the heating of the solid material for sublimation. Preferably, for precipitation from the gas phase and especially in terms of sublimation, several MALDI matrix substances are preferably used in parallel or sequentially. The MALDI matrix substances can be used for making different MALDI matrix points. But it is also conceivable that a MALDI matrix point shows a substructure, for example partial points existing separately from each other, which are each made of a different MALDI matrix substance. A substructure can also consist of concentric annuli, each consisting of a different MALDI matrix substance.

Preferably, a shaped body, a so-called mask with through holes, covers the sample carrier during the precipitation from the gas phase, especially preferred by way of sublimation. The MALDI matrix substance only precipitates itself on the surface formation in the area of these holes and forms a MALDI matrix point or partial point there. This mask can have any number of holes, which can have any shape. For example, the holes can be round, rectangular, square, triangular or oval, to name just a few of the possible shapes. The shape can be used to differentiate the respective MALDI matrix substances on a surface formation. The surface formation can also be initially covered by several masks, which are then removed one by one in order to, for example, apply different MALDI matrix substances to different areas of the surface formation according to the invention.

In particularly preferred embodiment of the present invention, the mask has further holes by means of which information can be transferred to the surface formation. In the area of these holes, the MALDI matrix substance precipitates itself on the surface formation, making the information visible there. Examples of information for the purpose of the invention would be the marking of the rows and columns of a grid, abbreviations for the MALDI matrix substance used, and alignment points that enable an exact alignment of the surface formation in the corresponding analysis device.

Preferably, the MALDI matrix points have an area of 1 μm²-10 mm². On such a surface, a drop of liquid can be deposited and preferably anchored such that it does not detach itself from the surface formation according to the invention even when hanging downwardly.

Furthermore, the MALDI matrix points should preferably be arranged along an exact grid, which allows for the easy control of metering and/or analysis machines. The MALDI matrix points can have any shape. Examples of possible shapes are rectangular, square, triangular or oval. The shape of the MALDI matrix points can be used for their differentiation, because the shape is recognisable, for example, under a microscope in the mass spectrometer during an analysis. For example, a certain shape can be assigned to a certain MALDI matrix substance. Furthermore, a MALDI matrix point preferably has a substructure. This substructure can consist of several partial points isolated from each other, which preferably each consist of a different MALDI matrix substance. The substructure can also have several concentric annuli. In particular, the embodiment consisting of several partial points is beneficial in that a single drop of a substance to be analysed, which is brought in contact with the MALDI matrix point, wets several partial points simultaneously and, for this reason, allows several different matrix substances to be examined at one MALDI matrix point.

Preferably, the MALDI matrix points or partial points represent areas that are more wettable than their surroundings and which are each completely surrounded by a less wettable, preferably ultraphobic area. Using this embodiment allows for the deposit of a drop of liquid at a very specific location and makes it possible to anchor it comparatively firmly there.

Preferably, the crystalline structure of the MALDI matrix points or partial points has a crystallite size <1 μ/m. This preferred embodiment of the present invention has the advantage, for example, that the MALDI matrix points of the substance to be tested are etched very well and evenly and/or that a very good signal is obtained.

For the purpose of the invention, ultraphobic means that the contact angle of a drop of water and/or oil lying on an ultraphobic surface is more than 1500, preferably more than 160° and ideally 170° with the roll-off angle not exceeding 10°. Roll-off angle means the angle of inclination of a basically planar but structured surface from the horizontal, at which a stationary drop of water and/or oil with a volume of 10 μl is moved due to gravity when the surface is inclined. Such ultraphobic surfaces are disclosed for example in WO 98/23549; WO 96/04123; WO 96/21523; WO 00/39369; WO 00/39368; WO 00/39239; WO 00/39051; WO 00/38845, and WO 96/34697, which are hereby incorporated as references and thus count as part of the disclosure.

In a preferred embodiment, the ultraphobic surface has a surface topography for which the local frequency of the individual Fourier components and their amplitude a(f) is expressed by the integral S(log(f))=a (f).f calculated between the integration limits log (f₁/μm⁻¹)=−3 and log (f₂/μm⁻¹)=3 is at least 0.3 and which consists of a hydrophobic or particularly an oleophobic material or are coated with a durable hydrophobic and/or particularly a durable oleophobic material. Such an ultraphobic surface is described in the international patent application WO 00/39249, which is hereby incorporated as a reference and thus counts as part of the disclosure.

The process according to the invention is particularly suitable for making surface formations with a multitude of MALDI matrix points. Therefore, this surface formation is also an object of the present invention.

In a preferred embodiment of the present invention, the surface formation is configured as a single use article. A surface formation with several layers and a first layer with an ultraphobic surface, and a carrier layer, with the first layer being applied reversibly to the carrier layer, and the maximum local flatness deviation of the surface formation being 100 μm, preferably <20 μm on a length of 100 mm, is particularly suitable for this embodiment.

This surface formation has the advantage of allowing the first layer with the ultraphobic surface to detach from the carrier layer after one or after several uses and to be replaced by a new first layer so that it is impossible for the first layer to be contaminated by previous experiments. The first layer with the ultraphobic surface is particularly economic when made to be disposable. As a result of the defined flatness according to the invention, the surface formation is guaranteed to be usable in all current mass spectrometers and/or optical analysis equipment.

In a preferred embodiment of the surface formation the first layer is adhered to the carrier layer.

Furthermore, an electrical contact preferably provided between the first layer and the carrier layer. This embodiment is particularly advantageous for mass spectroscopic analyses.

While the surface formation, according to the invention, has diverse uses, it is especially suitable for mass spectroscopic and/or optical analyses.

A further object of the present invention is a surface formation which is stable for a long time with at least one MALDI matrix point, characterised in that it is surrounded by a hollow body containing a vacuum and consisting of a material impervious to water vapour.

Reference is made to the above disclosure with respect to the surface formation and the MALDI matrix points.

According to the invention, the surface formation is completely surrounded by a hollow body containing a vacuum, preferably at a pressure of <100 mbar.

Furthermore, in accordance with the invention, the hollow body is made of a gas-proof material, which is especially impervious to water vapour.

The hollow body should preferably be impervious to light.

In addition, the hollow body should ideally be made of a plastic film that is sealed on at least one side. First and foremost, the plastic film should be a gas barrier layer, especially a barrier layer for water vapour. Preferably, this barrier layer is made of aluminium.

The surface formation according to the invention has the advantage that in particular the MALDI matrix points on the surface formation are subject to no or only minimal aging over a time period of at least several months.

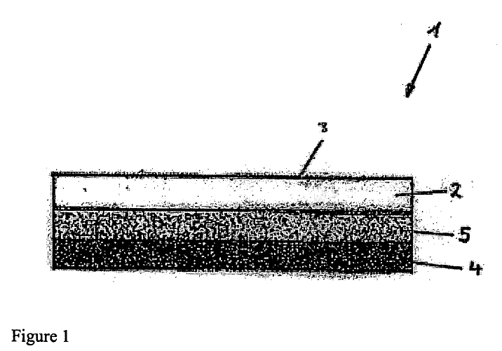

In the following, the invention is explained by means of Example 1 and the FIGS. 1-5. These explanations are merely exemplary and do not limit the general notions of the invention.

FIG. 1 shows a cross-section of a surface formation according to the invention with several layers.

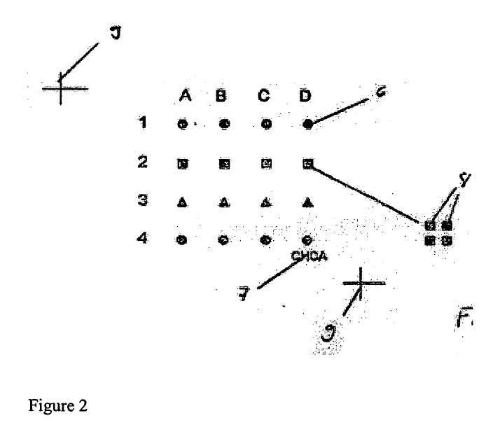

FIG. 2 shows the surface of a surface formation according to the invention.

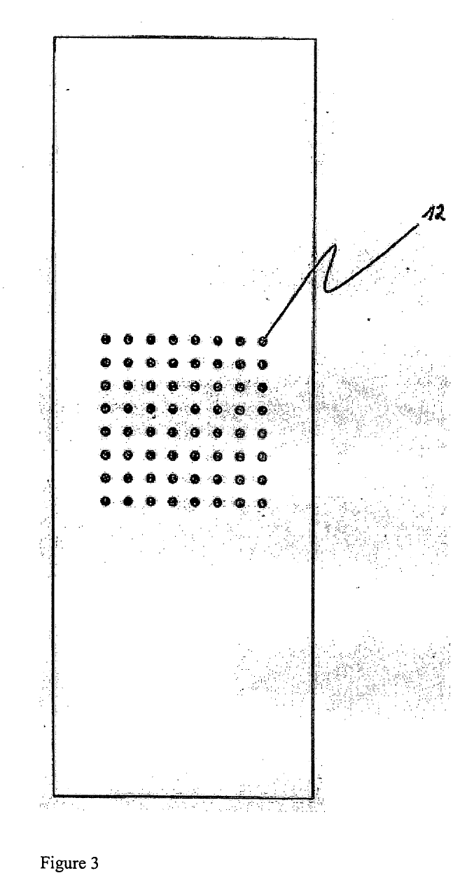

FIG. 3 shows a mask for the execution of the process for manufacturing a surface formation according to the invention.

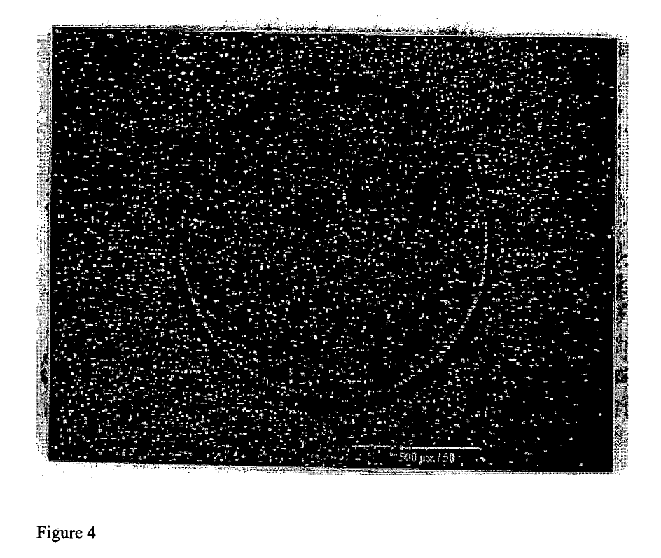

FIG. 4 shows a MALDI matrix point obtainable with the process according to the invention.

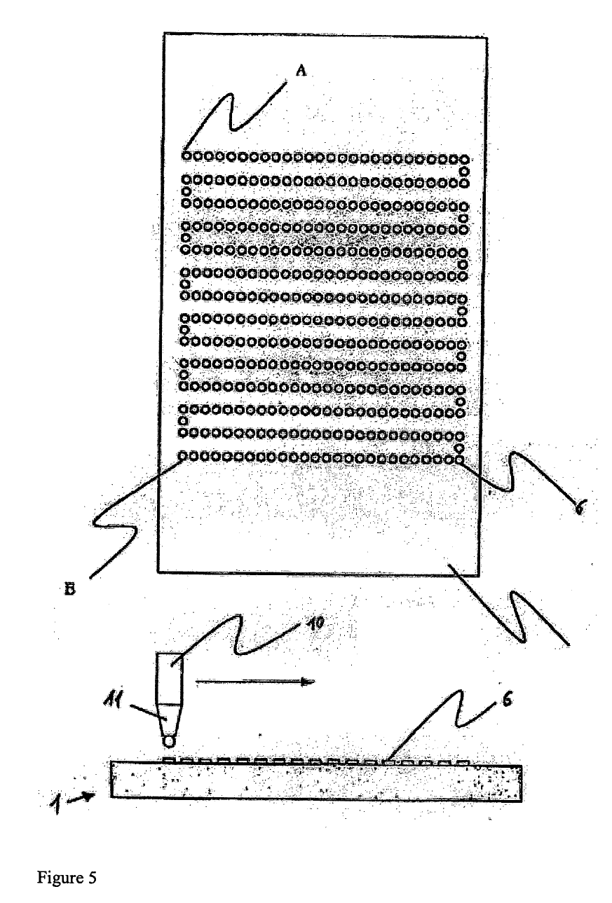

FIG. 5 shows the use of the surface formation according to the invention for the connection of liquid chromatography and MALDI examinations.

### EXAMPLE 1

A sample carrier was made as follows:

A roller-polished Al sheet (99.9%) with a surface of 26×76 mm² and a thickness of 0.15 mm was degreased at room temperature firstly with chloroform (p.a.) and afterwards with aqueous NaOH (5 g/l) at 50° C. for 20s.

Thereafter it was prepickled in H₃PO₄ (100 g/l) for 20s, rinsed in distilled water for 30s and electrochemically pickled for 90s in a mixture of HCl/H₃BO₃ (4 g/l each) at 35° C. and 120 mA/cm² at 35 V alternating current.

After rinsing in water for 30s and alkaline rinsing in aqueous NaOH (5 g/l) for another 30s, it was rinsed again in distilled water for 30s and afterwards anodically oxidised for 90s in H₂SO₄ (200 g/l) at 25° C. with 30 mA/cm² at 50 V direct current.

Afterwards, it was rinsed for 30s in distilled water, then for 60s at 40° C. in NaHCO₃ (20 g/l), and then again for 30s in distilled water and dried in the drying cabinet for 1 hour at 80° C.

The sheet thus treated was coated with an approx. 50 m thick layer of gold using cathodic evaporation in high vacuum. Finally, the sample was coated with a monolayer by immersion for 24 hours in a solution of the thiol CF₃—(CF₂)₇—(CH₂)₂—SH in benzotrifluoride (p.a. 1 g/l) at room temperature in a closed container, rinsed afterwards with benzotrifluoride (p.a.) and dried.

The surface has a static contact angle of 178° for water. On inclining the surface by <2°, a drop of water with a volume of 10 μl rolls away.

Matrix points were sublimated on the sample carrier as follows:

In a high-vacuum evaporation system (Edwards E306), 0.5 g of α-cyano-4-hydroxycoumarin acid are filled into a heatable quartz crucible with an opening at the top of 10 mm in diameter. A sample carrier is mounted at a spacing of 150 mm, and covered with a mask according to FIG. 3. After pumping off to a base pressure of <10⁻⁵ mbar, the quartz crucible is heated using tungsten coils located on the outside.

The temperature of the solid material is regulated to 180° C. in the powder packing by means of a thermocouple. The layer thickness of the deposited film made of α-cyano-4-hydroxycoumarin acid is determined using a quartz crystal layer thickness-measuring device, which is calibrated beforehand by means of absolute layer thickness testing (e.g. atomic force microscope). Sublimation is stopped at a layer thickness of 1 μm.

The MALDI matrix points obtained are shown as an example in FIG. 4. A mass spectrum of this MALDI matrix point was obtained as follows:

MALDI mass spectrum of the simple protonised peptides (1-7): human angiotensin I and II, substance P-methylester, neurotensin (amino acids 1-11), neurotensin, ACTH (amino acids 1-17) and ACTH (amino acids 18-39), recorded from a prepared MALDI matrix point with a diameter of 800 μm. The peptides were prepared for the mass spectrometric analysis as follows: 0.5 μl of an aqueous solution, containing 5 fmol of the peptides 1-6, 1 fmol of the peptide 7 as well as one per cent by volume trifluoroacetic acid and 1 mM of the non-ionic detergent n-octyl-β-D-glucopyranoside, were pipetted onto the MALDI matrix point. After the solvent was completely evaporated, the sample thus prepared was washed once by dipping the whole sample carrier into 0.1% trifluoroacetic acid for 2 seconds and by afterwards holding it immediately in a stream of nitrogen gas for 10 seconds to remove any remaining liquid. The mass spectrum of positive molecular ions was recorded in a MALDI time of flight (TOF) mass spectrometer produced by Bruker Daltonik, Bremen, (Scout-MTP Autoflex) in reflector operation mode and with time-delayed ion extractions (delay time: 70 nanoseconds) and 20 kV acceleration potential. 100 single-shot spectra were summed to improve the signal-to-noise ratio.

The result is shown in the following graphic.

[image]

FIGS. 1 to 5 are described in the following.

FIG. 1 shows the surface formation 1 which consists of a first layer 2 with an ultraphobic surface 3 and a carrier layer 4. The first layer 2 is fixed onto the carrier layer using a glue layer 5. Those of ordinary skill in the art will notice that the glue layer 5 does not necessarily have to be present. The glue layer 5 consists of an electrically conductive material, so that there is an electrical contact between the first layer 2 and the carrier material.

FIG. 2 shows a surface formation according to the invention on which several MALDI matrix points 6 were sublimated in the form of a grid. The MALDI matrix points in rows 1-4 have different shapes, symbolising to the user that different MALDI matrix substances were used in each row. The MADI matrix point 2D is shown enlarged to show that it consists of four partial points 8. These partial points 8 are built up from different MALDI matrix substances so that four different analyses can be made at this MALDI matrix point. In addition, a marking 7 is applied to the surface formation beneath the MALDI matrix point 4D, which informs the user about the MALDI matrix substance used in this row. In addition, the surface formation shows two centering crosses 9.

The marking and the centering crosses are sublimated, like the MALDI matrix points, onto the surface formation by putting a mask over the surface material having corresponding holes. The person of ordinary skill in the art will recognise that rows 1-4 of the grid were produced one at a time.

FIG. 3 shows the mask with 8×8 openings 12 with a diameter of 0.8 mm spaced at 2.25 mm from the centre points (not to scale).

FIG. 4 shows a light-microscopic recording of a matrix spot that was obtained with the mask in FIG. 3.

The exceptionally beneficial use of the sample carrier according to the invention for the connection of liquid chromatography and MALDI examination (LC-MALDI connection) is shown in FIG. 5.

Often, substance mixtures are initially chromatographically separated before a MALDI examination. An example would be mixtures of peptides created by an enzymatic (e.g. trypsin) peptide reduction. After the chromatographic separation the fractions of the eluate are then applied to the MALDI matrix points 6 of the sample carrier.

In the present example, MALDI matrix points 6, lying very close to each other, but being completely surrounded by an ultraphobic area, are used. Preferably, the spacings of the MALDI matrix points are (measured from centre point to centre point) 1.5 times their diameter.

By using MALDI matrix points that were produced by means of sublimation via masks, their location and size are defined very precisely. This will allow the liquid to continually run out of the outlet opening 11 of the LC column 10 for application to the MALDI matrix points. Separate collection of the fractions in containers with pipetting onto the matrix points afterwards or a discontinuous production of fractions by pausing the outlet opening over a MALDI matrix point and quickly moving on to the next MALDI matrix point afterwards is not required.

The sample carrier 1 is moved at a constant speed under the outlet opening 11. Every MALDI matrix point now captures a constant volume of liquid that is dispensed from the outlet opening. The process begins at point A and continues sinuously across all points to point E. The MALDI matrix points will then contain the whole eluate of the chromatography on the MALDI matrix points A to E in fractions according to the constant volume that each MALDI matrix point accepts. Changing the speed used to move the carrier 1 under the outlet opening 11 can change this volume.

1. Process for manufacturing a surface formation, preferably a sample carrier, with a multitude of MALDI matrix points, characterised in that the MALDI matrix points are applied to the sample carrier by precipitation of a MALDI matrix substance from the gas phase, preferably by sublimation.

2. Process according to claim 1, characterised in that a plate covers the sample carrier during the precipitation from the gas phase, which plate has through holes whose cross-sectional area corresponds to the cross-sectional area of the respective MALDI matrix points.

3. Process according to claim 2, characterised in that the plate has at least one further through hole by means of which information is transferred to the sample carrier by precipitation of the MALDI matrix substance from the gas phase.

4. Process according to claim 3, characterised in that the information comprises, for example, the composition of the MALDI matrix substance and/or alignment points.

5. Process according to claim 1, characterised in that the MALDI matrix points are arranged along a grid.

6. Process according to claim 1, characterised in that the MALDI matrix points have substructures.

7. Process according to claim 6, characterised in that the MALDI matrix points are separated into several partial points, preferably isolated from one another.

8. Process according to claim 1, characterised in that different MALDI matrix substances are applied to a sample carrier.

9. Process according to claim 8, characterised in that at least several MALDI matrix points or partial points each consisting of one MALDI matrix substance are built up.

10. Process according to claim 1, characterised in that α-cyano-4-hydroxycoumarin acid is used as a MALDI matrix substance.

11. Process according to claim 1, characterised in that the sample carrier has an ultraphobic surface.

12. Process according to claim 11, characterised in that the MALDI matrix points or partial points represent hydrophilic areas which are completely surrounded by ultraphobic areas.

13. Surface formation obtainable with a process according to claim 1.

14. Surface formation according to claim 13, characterised in that it has several layers.

15. Surface formation (1) according to claim 14, characterised in that it has a first layer (2) with an ultraphobic surface (3) and a carrier layer (4).

16. Surface formation according to claim 15, characterised in that the first layer (2) is applied reversibly on a carrier layer (4) and the maximum local flatness deviation of the surface formation on a length of 100 mm is <100 μm.

17. Surface formation according to claim 16, characterised in that the first layer (2) is glued to the carrier layer (4).

18. Surface formation according to claim 16, characterised in that there is an electrical contact between the first layer (2) and the carrier layer (4).

19. Long-time stable surface formation with at least one MALDI matrix point, characterised in that it is surrounded by a hollow body containing a vacuum and consisting of material impervious to water vapour and, preferably, impervious to light.

20. Surface formation according to claim 19, characterised in that it has additional biological material on the MALDI matrix point.
